(19) United States
(12) Patent Application Publication
(10) Pub. No.: US 2011/0144206 A1
(43) Pub. Date: Jun. 16, 2011

(54) USE OF A COX-2 INHIBITOR FOR THE TREATMENT OF A COX-2 DEPENDENT DISORDER IN A PATIENT NOT CARRYING HLA ALLELES ASSOCIATED WITH HEPATOTOXICITY

(76) Inventors: Steven Lewitzky, Boston, MA (US); Joanne Meyer, Framingham, MA (US); Charles Paulding, Groton, MA (US); Jonathan B Singer, Somerville, MA (US)

(21) Appl. No.: 13/060,137

(22) PCT Filed: Aug. 20, 2009
(86) PCT No.: PCT/US09/54415

§ 371 Date: Feb. 22, 2011

Related U.S. Application Data
(60) Provisional application No. 61/091,081, filed on Aug. 22, 2008.
(60) Provisional application No. 61/211,698, filed on Apr. 2, 2009.

(51) Int. Cl. A61K 31/196; C12Q 1/68; C40B 30/04; G01N 33/50; C07H 21/00; A61P 35/00; A61P 25/00; A61P 25/28; A61P 11/06; A61P 37/02; A61P 25/06

(52) U.S. Cl. 514/567; 435/6.11; 435/6.12; 506/9; 436/94; 536/24.31

(57) ABSTRACT

This disclosure relates to a method of determining the presence of at least one HLA allele, preferably selected from the group consisting of DQA1*0102, DRB1*1501, DQB1*0602 and DRB5*0101 to assess whether a patient is at risk for developing hepatotoxicity upon administration of the COX-2 inhibitor lumiracoxib. Also disclosed is the use of a kit for carrying out this method. The disclosure also relates to a method of treating cyclooxygenase-2 dependent disorders with lumiracoxib in a subject that is not a carrier of one or more HLA alleles, preferably selected from the group consisting of DQA1*0102, DRB1*1501, DQB1*0602 and DRB5*0101.

### BACKGROUND OF THE DISCLOSURE

The present disclosure relates, inter alia, to methods of predicting the risk of a patient for developing hepatotoxicity upon administration of the COX-2 inhibitor lumiracoxib by determining the presence of Human Leukocyte Antigen (HLA) alleles.

Nonsteroidal anti-inflammatory drugs (NSAIDS) are widely used and well-established treatments for chronic pain in osteoarthritis and pain and inflammation in rheumatoid arthritis. The mechanism of action for all NSAIDS is attributed to their blockade of prostaglandin synthesis by inhibition of the cyclooxygenase pathway. Inhibition of prostaglandin production, however, causes common side effects, such as gastric irritation, vascular constriction and renal impairment.

Lumiracoxib (2-[(2-chloro-6-fluorophenyl)amino]-5-methyl-benzeneacetic acid) (C₁₅H₁₃NO₂ClF) is a potent and selective inhibitor of cyclooxygenase (COX)-2. Lumiracoxib is as effective as non-selective, non-steroidal NSAIDs in relieving the signs and symptoms of osteoarthritis, pain and rheumatoid arthritis. It has good oral bioavailability and rapid absorption, reaching maximum plasma concentrations two hours after dosing (C. M. Rordorff et al., Clin. Pharmacokinet., 44(12), 1247-1266 (2005)). Despite its short elimination half-life of four hours from plasma, lumiracoxib is effectively distributed to inflamed tissues and retained for up to 24 hours (A. Buvanendran and R. Barkin, Drugs of Today, 43(3), 137-147 (2007)). Compared to prior NSAIDS, lumiracoxib also has an improved tolerability profile, especially for the gastrointestinal system (Y. Yuan and R. Hunt, Current Pharm. Design, 13, 2237-2247 (2007)).

While generally safe and well-tolerated, some patients treated with lumiracoxib experience elevated alanine aminotransferase (ALT) and/or aspartate aminotransferase (AST) enzyme levels in the blood. This increase in ALT/AST levels may result in a rare but serious side effect of hepatotoxicity (Y. Li et al., Drug Metab. Disp., 36, 469-473 (2008)).

Despite the advancements in genomic knowledge, definitive and reproducible insights into how genetic variants relate to a patient's adverse drug reaction are lacking. Defining how particular genetic alleles are associated with a patient's susceptibility to an adverse drug reaction provides an opportunity for the improved and safer treatment of disease. There is need in the art for a useful approach to assess a patient's susceptibility for developing hepatotoxicity in response to lumiracoxib.

### SUMMARY OF THE DISCLOSURE

The present disclosure provides methods of predicting the risk of a patient for developing hepatotoxicity in response to the selective COX-2 inhibitor lumiracoxib. It is discovered that HLA alleles DQA1*0102, DRB1*1501, DQB1*0602, and DRB5*0101 are highly associated with hepatotoxicity induced by lumiracoxib. As such, the HLA alleles DQA1*0102, DRB1*1501, DQB1*0602 and DRB5*0101 can be used as individual biomarkers for predicting a patient's risk for developing hepatotoxicity upon treatment with lumiracoxib.

Accordingly, the present application provides methods of assessing the risk of a patient for developing hepatotoxicity in response to the administration of lumiracoxib. Once risk has been ascertained, the patient may be treated (or treatment continued, or treatment proceed with a dosage increase) with lumiracoxib (in the case of a low or non-risk determination), or the patient may not be treated (or treatment discontinued, or treatment proceed with a lowered dose) with lumiracoxib (in the case of a high risk determination). As such, the instant disclosure provides methods of treatment with lumiracoxib, wherein said treatment protocol is determined based on the analysis of HLA alleles DQA1*0102, DRB1*1501, DQB1*0602, and DRB5*0101, and the risk of hepatotoxicity. Assessing the risk of a patient for developing hepatotoxicity in response to lumiracoxib can be accomplished by determining the presence or absence of at least one HLA allele selected from the group consisting of DQA1*0102, DRB1*1501, DQB1*0602, or DRB5*0101, wherein the presence of the HLA allele is indicative of risk for hepatotoxicity. Preferably, presence or absence of HLA allele DQA1*0102, is indicative of risk for hepatotoxicity.

A genetic allele can be detected by direct detection of regions/nucleotides within the allele using genomic DNAs prepared from biological samples. While these biomarkers are identified from blood as described in the examples, the sample from which they may be detected is not limited to blood but may be detected in other types of samples such as buffy coat, serum, plasma, lymph, urine, tear, saliva, cerebrospinal fluid, buccal swabs, sputum, or tissue. It also can be determined by detecting an equivalent genetic marker of the allele, which can be, e.g., an SNP (single nucleotide polymorphism), a microsatellite marker or other kinds of genetic polymorphisms. In other words, the presence of a genetic marker on the same haplotype as the HLA allele DQA1*0102, DRB1*1501, DQB1*0602 or DRB5*0101 allele, rather than the allele per se, is indicative of a patient's risk for developing a hepatotoxic reaction. Exemplary equivalent genetic markers of the HLA haplotypes include the single nucleotide polymorphisms identified as rs3131294, rs3129868, rs9270986, rs3129900, and rs3135365 by the NCBI Database (http://www.ncbi.nlm.nih.gov/).

Also within the scope of this disclosure is a method for determining whether a patient carries HLA allele DQA1*0102 to assess the patient's risk of developing hepatotoxicity in response to lumiracoxib. This method comprises detecting presence of the HLA allele DQA1*0102 from a biological sample obtained from the subject. This region may be detected by methods known in the art, e.g., Sequence Specific Oligonucleotide (SSO) hybridization coupled with the Luminex xMAP® technology Sequence Specific Primer (SSP) typing, Sequence Based Typing (SBT).

In alternate embodiments, these methods may be used to determine whether the patient carries at least one of HLA allele subtypes DRB1*1501, DQB1*0602 or DRB5*0101.

The present disclosure also relates to a method of identifying or predicting the predisposition to hepatotoxicity or the risk of developing hepatotoxicity and/or elevated ALT or AST in a subject treated with lumiracoxib comprising assaying a biological sample obtained from a subject (e.g., human) for the presence of at least one HLA allele, wherein the presence of said at least one HLA allele is indicative of the presence or increased prediction of hepatotoxicity and/or elevated ALT or AST or an increased risk of developing hepatotoxicity in said subject, and wherein the absence of said at least one HLA allele is indicative of the absence or decreased prediction of hepatotoxicity or a decreased risk of developing hepatotoxicity in said subject.
Preferably, the HLA allele is selected from the group consisting of DQA1*0102, DRB1*1501, DQB1*0602 and DRB5*0101.

Other objects, features, advantages and aspects of the present disclosure will become apparent to those skilled in the art from the following description and appended claims. It should be understood, however, that the following description, appended claims, and specific examples, while indicating preferred embodiments of the disclosure, are given by way of illustration only. Various changes and modifications within the spirit and scope of the disclosed subject matter will become readily apparent to those skilled in the art from reading the following.

### DESCRIPTION OF THE FIGURES

FIG. 1 Whole genome association results for all SNPs (p-values graphed by genomic location).

FIG. 2 Observed and expected distributions of −log 10 (p-value) from whole genome association study.

FIG. 3 Mean peak ULN ALT/AST levels of cases for DQA1*0102 carriers and non-carriers.

FIG. 4 Kaplan-Meier incidence estimates calculated for the DQA1*0102 carriers and non-carriers for >3xULN.

FIG. 5 Kaplan-Meier incidence estimates calculated for the DQA1*0102 carriers and non-carriers for >5xULN.

### DETAILED DESCRIPTION OF THE DISCLOSURE

Listed below are definitions of various terms used to describe the genetic markers of the present disclosure. These definitions apply to the terms as they are used throughout the specification unless they are otherwise limited in specific instances either individually or part of a larger group.

The term “lumiracoxib” as used herein refers to the selective COX-2 inhibitor 2-[(2-chloro-6-fluorophenyl)amino]-5-methyl-benzeneacetic acid) (C₁₅H₁₃NO₂ClF) and includes, as appropriate, pharmaceutically acceptable salts and esters thereof. One of skill in the art would recognize that lumiracoxib may be administered to a patient as a pharmaceutical composition, a medicament or other suitable dosage form.

“Genetic marker” and “Biomarker” in the context of the present disclosure refers to HLA allele DQA1*0102, DRB1*1501, DQB1*0602 or DRB5*0101 and its related genetic products (i.e. proteins or polypeptides, mRNA) which are present in a sample taken from subjects having a risk of developing hepatotoxicity in response to the selective COX-2 inhibitor lumiracoxib.

“Proteins or polypeptides” of the present disclosure are contemplated to include any fragments thereof, in particular, immunologically detectable fragments. One of skill in the art would recognize that damaged proteins may become degraded or cleaved into fragments. Additionally, certain proteins or polypeptides are synthesized in an inactive form, which may be subsequently activated by proteolysis. Such fragments of a particular protein may be detected as a surrogate for the protein itself.

The term “sample” as used herein refers to a sample from a subject obtained for the purpose of identification, diagnosis, prediction, or monitoring. In certain aspects of the disclosure such a sample may be obtained for the purpose of predicting the risk of a patient for developing hepatotoxicity upon administration of the selective COX-2 inhibitor lumiracoxib. Preferred test samples include blood, blood-derived product (such as buffy coat, serum, and plasma), lymph, urine, tear, saliva, cerebrospinal fluid, buccal swabs, sputum, or tissue samples. In addition, one of skill in the art would realize that some test samples would be more readily analyzed following a fractionation or purification procedure, for example, isolation of DNA from whole blood.

The phrase “probability of developing hepatotoxicity” as used herein refers to methods by which the skilled artisan can predict the risk of a subject for developing hepatotoxicity in response to administration of the selective COX-2 inhibitor lumiracoxib. It does not refer to the ability to predict the development of hepatotoxicity with 100% accuracy. Instead, the skilled artisan will understand that it refers to an increased probability that hepatotoxicity will develop.

A patient has a “risk” or “predisposition” for developing hepatotoxicity upon administration of the COX-2 inhibitor lumiracoxib if the probability of the patient to develop hepatotoxicity is higher than the probability of the general population to develop hepatotoxicity. The probability of the patient to develop hepatotoxicity in response to administration of lumiracoxib is at least about 1.5 fold, more preferably at least about 2 fold, still more preferably at least about 3, 4, 5, 6, 7, 8 or 9 fold, and most preferably at least about 10 fold higher than the probability of the general population to develop hepatotoxicity in response to lumiracoxib. The probability can be determined by any method known in the art.

The term “sensitivity” of the genetic marker of the present disclosure is the percentage of treated patients with hepatotoxicity and/or elevations in ALT and/or AST that possess the genetic marker. The sensitivity of a risk factor is preferably at least about 40%, more preferably at least about 50%, 60%, 70%, 80%, 85% or 90%. Most preferably, the sensitivity is at least 95% or higher. Individuals suffering from hepatotoxicity as a direct result from the treatment with lumiracoxib and not detected by the assay are “false negatives”. Subjects who are not hepatotoxic and who test negative in the assay, are termed “true negatives.” The “specificity” of a diagnostic assay is 1 minus the false positive rate, where the “false positive rate” is defined as the proportion of those without hepatotoxicity associated to the treatment who test positive. While a particular diagnostic method may not provide a definitive diagnosis of a condition, it suffices if the method provides a positive indication that aids in diagnosis.

An “equivalent genetic marker” of an allele of interest refers to a genetic marker that is correlated to the allele of interest, i.e., it displays linkage disequilibrium with the allele of interest.

The term “probe” used herein refers to any substance useful for specifically detecting another substance related to HLA allele DQA1*0102, DRB1*1501, DQB1*0602 or DRB5*0101. A probe can be an oligonucleotide or conjugated oligonucleotide that specifically hybridizes to a particular region within HLA allele DQA1*0102, DRB1*1501, DQB1*0602 or DRB5*0101. The conjugated oligonucleotide refers to an oligonucleotide covalently bound to chromophore or molecules containing a ligand (e.g., an antigen), which is highly specific to a receptor molecule (e.g., an antibody specific to the antigen). The probe can also be a PCR primer, together with another primer, for amplifying a particular region within HLA allele DQA1*0102, DRB1*1501, DQB1*0602 or DRB5*0101. Further, the probe can be an antibody that specifically recognizes at least one of HLA alleles DQA1*0102, DRB1*1501, DQB1*0602 and DRB5*0101 or a protein product of the allele.

As described herein above, the present disclosure relates to a method of identifying or predicting the predisposition to hepatotoxicity or the risk of developing hepatotoxicity and/or elevated ALT or AST in a subject treated with lumiracoxib comprising assaying a biological sample obtained from a subject (e.g., human) for the presence of at least one HLA allele, wherein the presence of said at least one HLA allele is indicative of the presence or increased prediction of hepatotoxicity and/or elevated ALT or AST or an increased risk of developing hepatotoxicity in said subject, and wherein the absence of said at least one HLA allele is indicative of the absence or decreased prediction of hepatotoxicity or a decreased risk of developing hepatotoxicity in said subject. Preferably, the HLA allele is selected from the group consisting of DQA1*0102, DRB1*1501, DQB1*0602 and DRB5*0101.

In addition, the present disclosure also relates to a method for predicting Hy's law cases (>3xULN ALT/AST and ≧2xULN serum bilirubin) upon administration of lumiracoxib comprising assaying a biological sample obtained from a subject for the presence of at least one HLA allele selected from the group of DQA1*0102, DRB1*1501, DRB5*0101 and DQB1*06 in said subject.

The biological sample is selected from the group of: blood, serum, plasma, urine, tear, saliva, cerebrospinal fluid, leukocyte sample or tissue sample or a combination thereof. The most preferred HLA allele for detection is DQA1*0102.
The relevant HLA alleles include but are not limited to:

i) HLA allele DQA1*0102, comprising DQA1*010201 (SEQUENCE ID No. 1), DQA1*010202 (SEQUENCE ID No. 2), DQA1*010203 (SEQUENCE ID No. 3), DQA1*010204 (SEQUENCE ID No 4). The amino acid sequence of DQA1*0102 is disclosed as SEQUENCE ID No. 5.

ii) HLA allele DRB1*1501, comprising DRB1*15010101, DRB1*15010102 (SEQUENCE ID No. 6), DRB1*150102 (SEQUENCE ID No. 7), DRB1*150103 (SEQUENCE ID No. 8), DRB1*150104 (SEQUENCE ID No. 9), DRB1*150105 (SEQUENCE ID No. 10) and DRB1*150106 (SEQUENCE ID No. 11). The amino acid sequence of DRB1*150101 (DRB1*15010101, *15010102) is disclosed as SEQUENCE ID No. 12.

iii) HLA allele DQB1*0602, comprising DQB1*060201 (SEQUENCE ID No. 13) and DQB1*060202 (SEQUENCE ID No. 14). The amino acid sequence of DQB1*060201 is disclosed as SEQUENCE ID No. 15.

iv) HLA allele DRB5*0101, comprising DRB5*010101 (SEQUENCE ID No. 16) and DRB5*010102 (SEQUENCE ID No. 17). The amino acid sequence of DRB5*010101 is disclosed as SEQUENCE ID No. 18.

The relevant information regarding the nucleic acid and amino acid sequences of the HLA alleles is accessible to those skilled in the art in well-known databases such as the IMGT/HLA Database website at http://www.ebi.ac.uk/imgt/hla/. The present disclosure also includes additional DQA1*0102, DRB1*1501, DQB1*0602 and DRB5*0101 as new alleles are discovered.

In addition, the present disclosure also describes a method for predicting Hy's law cases (>3xULN ALT/AST and ≧2xULN serum bilirubin) upon administration of lumiracoxib comprising assaying a biological sample obtained from a subject for the presence of at least one HLA allele selected from the group of DQA1*0102, DRB1*1501, DRB5*0101 and DQB1*0602 in said subject.

In addition to the specific HLA alleles per se, genetic markers that are correlated to any of the specific alleles can be used to predict a patient's risk of developing hepatotoxicity in response to administration of lumiracoxib. Thus, genetic markers that associate, i.e., due to linkage disequilibrium or genetic linkage, may be used to indicate the presence of an HLA of interest. Consequently, the presence of these markers (equivalent genetic markers) is indicative of the presence of the HLA allele of interest, which in turn is indicative of a risk for hepatotoxicity. The HLA DQA1*0102 and DRB1*1501 haplotypes, for example, are indicated by the presence of an equivalent genetic marker such as the single nucleotide polymorphisms identified as rs3131294, rs3129868, rs9270986, rs3129900, and rs3135365 by the NCBI data base.

The equivalent genetic marker can be any marker, including an HLA allele, a microsatellite marker, and a SNP marker. Preferably, the useful equivalent genetic markers are about 200 kb or less from the HLA allele of interest. More preferably, the equivalent genetic markers are 100 kb or less from the HLA allele of interest.

Numerous methods and devices are well known to the skilled artisan to detect the presence of the particular genetic marker of the present disclosure. The presence of an HLA allele genetic marker can be determined by direct detection of that marker or particular regions within it. Genomic DNAs for allele detection can be prepared from a patient biological sample by methods well known in the art, e.g., PUREGENE DNA® purification system from Gentra Systems (Qiagen, CA). Detection of a region within a genetic marker of interest includes examining the nucleotide(s) located at either the sense or the anti-sense strand within that region.

Methods known in the art can be used to detect a particular region, e.g., sequence specific primer PCR (SSP) typing, sequence specific oligonucleotide (SSO) typing, or sequence based typing (SBT). The term “presence of the genetic marker” refers to the presence or amount of a specific gene including, but not limited to, mRNA, cDNA or the polypeptide expression product of a specific gene. Similarly, the equivalent genetic markers can be detected by any methods known in the art.

In addition, the presence of at least one HLA allele selected from the group consisting of DQA1*0102, DRB1*1501, DQB1*0602 and DRB5*0101 may be detected from genomic DNA obtained from PCR using sequence-specific probes, e.g., hydrolysis probes from Taqman, Beacons, Scorpions; or hybridization probes. For the detection, sequence specific probes are designed such that they specifically bind to the genomic DNA for the HLA alleles of interest. These probes may be labeled for direct detection or contacted by a second, detectable molecule that specifically binds to the probe. The PCR products also can be detected by DNA-binding agents.

Said PCR products can then be subsequently sequenced by any DNA sequencing method available in the art.
Alternatively the presence of said HLA alleles can be detected by sequencing using any sequencing methods such as, but not limited to, Sanger-based sequencing, direct sequencing or new or next generation sequencing (Shendure J. and Ji, H., Nature Biotechnology (1998), Vol. 26, Nr 10, pages 1135-1145)
In one embodiment, the presence of at least one HLA allele selected from the group consisting of DQA1*0102, DRB1*1501, DQB1*0602 and DRB5*0101 is detected using a hybridization assay. In a hybridization assay, the presence or absence of the genetic marker is determined based on the ability of the nucleic acid from the sample to hybridize to a complementary nucleic acid molecule, e.g., an oligonucleotide probe. A variety of hybridization assays are available. In some, hybridization of a probe to the sequence of interest is detected directly by visualizing a bound probe, e.g., a Northern or Southern assay. In these assays, DNA (Southern) or RNA (Northern) is isolated. The DNA or RNA is then cleaved with a series of restriction enzymes that cleave infrequently in the genome and not near any of the markers being assayed. The DNA or RNA is then separated, e.g., on an agarose gel, and transferred to a membrane. A labeled probe or probes, e.g., by incorporating a radionucleotide or binding agent (e.g., SYBR® Green), is allowed to contact the membrane under low-, medium- or high-stringency conditions. Unbound probe is removed and the presence of binding is detected by visualizing the labeled probe.
Various methods of assaying, detecting, measuring, identifying and/or determining the alleles are known in the art. Such methods include, but are not limited to, e.g., DNA amplification techniques such as PCR and variants thereof, direct sequencing, Sequence-Specific Oligonucleotide hybridization (SSO), Sequence Specific Primer typing (SSP), or Sequence Based typing (SBT).

Sequence-Specific Oligonucleotide (SSO) typing uses PCR target amplification, hybridization of PCR products to a panel of immobilized sequence-specific oligonucleotides on the beads, detection of probe-bound amplified product by color formation followed by data analysis.

Those skilled in the art would understand that the described Sequence-Specific Oligonucleotide (SSO) hybridization may be performed using various commercially available kits, such as the LABType® SSO DQA1/DQB1 Typing Test kits provided by One Lambda, Inc. (Canoga Park, Calif.) or Lifecodes HLA-DQA Typing Kit (Tepnel Life Sciences Corp.) coupled with Luminex® technology (Luminex, Corporation, TX). LABType® SSO is a reverse SSO (rSSO) DNA typing solution that uses sequence-specific oligonucleotide (SSO) probes and color-coded microspheres to identify HLA alleles. The target DNA is amplified by polymerase chain reactions (PCR) and then hybridized with the bead probe array. The assay takes place in a single well of a 96-well PCR plate; thus, 96 samples can be processed at one time.

Sequence Specific Primers (SSP) typing is a PCR based technique which uses sequence specific primers for DNA based HLA typing. The SSP method is based on the principle that only primers with completely matched sequences to the target sequences result in amplified products under controlled PCR conditions. Allele sequence-specific primer pairs are designed to selectively amplify target sequences which are specific to a single allele or group of alleles. PCR products can be visualized on agarose gel. Control primer pairs that matches non-allelic sequences present in all samples act as an internal PCR control to verify the efficiency of the PCR amplification. Those skilled in the art would understand that high resolution genotyping with the described sequence-specific primer typing may be performed using various commercially available kits, such as the Olerup SSP™ kits (Qiagen, CA) or (Invitrogen) or Allset and ™ Gold DQA1 Low resolution SSP (Invitrogen).

Sequence Based Typing is based on PCR target amplification, followed by sequencing of the PCR products and data analysis.

In another embodiment, the presence of at least one HLA allele selected from the group consisting of DQA1*0102, DRB1*1501, DQB1*0602 and DRB5*0101 is determined by measuring RNA levels. The HLA allele of interest may be detected using a PCR-based assay or reverse-transcriptase PCR (RT-PCR). In RT-PCR, RNA is enzymatically converted to cDNA using a reverse-transcriptase enzyme. The cDNA is then used as a template for a PCR reaction. PCR products can be detected by any suitable method including, but not limited to, gel electrophoresis and staining with a DNA-specific stain or hybridization to a labeled probe. In yet another aspect, the quantitative RT-PCR with standardized mixtures of competitive templates can be utilized.

In another embodiment, the presence of at least one HLA allele DQA1*0102, DRB1*1501, DQB1*0602 or DRB5*0101 is determined by measuring polypeptide gene expression products. In a preferred aspect of the disclosure, gene expression is measured by identifying the amount of one or more polypeptides encoded by one of the genes. The present subject matter is not limited by the method in which gene expression is detected or measured.

In yet another embodiment, the presence of at least one HLA allele DQA1*0102, DRB1*1501, DQB1*0602 or DRB5*0101 is determined by detecting protein or polypeptide expression product encoded by one of the genes using any method known in the art. With regard to polypeptides or proteins in samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

The presence or amount of a protein or polypeptide is generally determined using specific antibodies and detecting specific binding. Any suitable immunoassay may be utilized, for example, enzyme-linked immunoassays (ELISA), radioimmunoassays (RIAs), competitive binding assays, and the like. Specific immunological binding of the antibody to the protein or polypeptide can be detected directly or indirectly. Direct labels include fluorescent or luminescent tags, metals, dyes, radionucleides, and the like, attached to the antibody. Indirect labels include various enzymes well known in the art, such as alkaline phosphatase, hydrogen peroxidase and the like.

The use of immobilized antibodies specific for the proteins or polypeptides is also contemplated by the present disclosure. The antibodies can be immobilized onto a variety of solid supports, such as magnetic or chromatographic matrix particles, the surface of an assay place (such as microtiter wells), pieces of a solid substrate material (such as plastic, nylon, paper), and the like. An assay strip can be prepared by coating the antibody or a plurality of antibodies in an array on solid support. This strip can then be dipped into the test sample and then processed quickly through washes and detection steps to generate a measurable signal, such as a colored spot.

One of skill in the art would recognize that the analysis of the gene of the present methods may be carried out separately or simultaneously while analyzing other genetic sequences. In another aspect of the present disclosure, an array is provided to which probes that correspond in sequence to gene products, e.g., cDNAs, mRNAs, cRNAs, polypeptides and fragments thereof, can be specifically hybridized or bound at a known position.

In another aspect, the present disclosure provides a method of using a kit containing at least one probe for the detection of HLA allele DQA1*0102, DRB1*1501, DQB1*0602 and/or DRB5*0101 in a subject to determine whether the subject is susceptible for developing hepatotoxicity upon administration of lumiracoxib. These probes can be oligonucleotide or conjugated oligonucleotide that specifically hybridizes to a particular region within the HLA allele genetic marker (DQA1*0102, DRB1*1501, DQB1*0602 or DRB5*0101); a PCR primer, together with another primer, for amplifying a particular region within said HLA allele genetic marker; an antibody recognizing HLA allele genetic marker and/or a protein product of said HLA allele genetic marker. Optionally, the kit can contain a probe that targets an internal control allele, which can be any allele presented in the general population. Detection of an internal control allele is designed to assure the performance of the kit.

In another aspect of the disclosure provides a method of treating a cyclooxygenase-2 dependent disorder in a subject comprising the steps of:
(i) receiving data regarding the presence in a biological sample obtained from said subject of at least an HLA allele being indicative of the presence or prediction of hepatotoxicity,
ii) administering lumiracoxib to the subject if said received data indicates that the subject is not a carrier of said HLA allele.
In an alternative embodiment, the disclosure also provides a method of treating a cyclooxygenase-2 dependent disorder in a subject comprising the steps of:
(i) assaying the presence in a biological sample obtained from said subject of at least one HLA allele indicative of the presence or prediction of hepatotoxicity,
ii) administering lumiracoxib to the subject if the subject is not a carrier of one or more said HLA alleles
Preferably, the subject is human, and the biological sample is selected from the group consisting of normal tissue, bodily fluid and a combination thereof.
In addition, one or more of said HLA allele(s) is selected from the group consisting of DQA1*0102, DRB1*1501, DQB1*0602 and DRB5*0101. Preferably the HLA allele is DQA1*0102.
The present disclosure provides also method of treating a cyclooxygenase-2 dependent disorder by administering lumiracoxib to a subject that has a reduced predisposition or risk of developing hepatoxicity in response to lumiracoxib, wherein said reduced predisposition or risk is identified by the method set forth hereinabove.
Alternatively, a method for treating COX-2 dependent disorders in a subject can also be performed by
i) assaying, or receiving data regarding the presence in a biological sample obtained from said subject of at least an equivalent genetic markers being indicative of the presence of an HLA allele indicative of the presence or prediction of hepatotoxicity and
ii) administering lumiracoxib to the subject if the subject is not a carrier of said equivalent genetic marker.
The present disclosure provides also method of treating a cyclooxygenase-2 dependent disorder by administering lumiracoxib to a subject that has a reduced predisposition or risk of developing hepatoxicity in response to lumiracoxib, wherein said reduced predisposition or risk is identified by the method set forth hereinabove.
Lumiracoxib is a compound of the class of 5-alkyl substituted 2-arylaminophenylacetic acids and derivatives which surprisingly inhibit COX-2 without significantly inhibiting COX-1. Such nonsteroidal antiinflammatory agents are surprisingly free of undesirable side effects usually associated with the classical nonsteroidal antiinflammatory agents, such as gastrointestinal and renal side effects, and are described in International Patent Application Publication No. WO99/11605, published 11 Mar. 1999.
COX-2 inhibitors such as lumiracoxib are particularly useful for the treatment of cyclooxygenase-2 dependent disorders in mammals, as for example recited in International Patent Application Publication No. WO 98/16227, published 23 Apr. 1998. Preferably, the cycloxygenase-2 dependent disorder is an inflammatory disorder, osteoarthritis (e.g., of the knee, hip, spine and shoulder), rheumatoid arthritis, refractory osteoarthritis, ankylosing spondylitis, gout, acute gout, dental pain, post-surgery dental pain, post-operative pain, orthopaedic surgery pain, low back pain, sore throat, post-herpetic neuralgia, herpes zoster, trigeminal neuralgia, visceral pain, musculoskeletal pain, fibromyalgia, dysmenorrhea, renal and biliary colic, migraine, headache, pain associated with cancer, pyresis, neurodegenerative diseases such as multiple sclerosis, Alzheimer's disease, osteoporosis, asthma, lupus and psoriasis, neoplasia particularly neoplasia that produce prostaglandins or express cyclooxygenase, including both benign and cancerous tumors, growths and polyps, in particular epithelium cell-derived neoplasia, skin, gastrointestinal, basal cell, squamous cell, colon, liver, bladder, pancreas, ovarian, prostate, cervical, lung or breast cancer or melanoma, angiogenesis-mediated ocular diseases including age-related macular degeneration, diabetic retinopathy, diabetic macular oedema.
Most preferably the cyclooxygenase-2 dependent disorder is selected from the group consisting of osteoarthritis (e.g., of the knee, hip, spine and shoulder), rheumatoid arthritis, refractory osteoarthritis, ankylosing spondylitis, low back pain, dental pain, post-surgery dental pain, visceral pain, musculoskeletal pain, post-herpetic neuralgia, herpes zoster, trigeminal neuralgia, fibromyalgia, dysmenorrhoea.
Lumiracoxib is to be administered at a dose from about 25 mg to about 1200 mg, preferably from about 100 mg to about 400 mg.
Lumiracoxib is preferably administered in the form of a tablet, as described in International Patent Application Publication No. WO 02/20090, published 14 Mar. 2002.
A tablet can have any dosage, preferably from about 25 mg to about 1200 mg. More preferably the tablet is containing from about 100 mg to about 400 mg, most preferably about 100 mg, about 200 mg or about 400 mg of lumiracoxib.
Other dosage forms can also be used, such as an oral liquid dosage form such as a drinkable solution or a parenteral dosage form, a topical dosage form or eye drops or any other ophthalmic formulations.
The dosage regimen for lumiracoxib is preferably, but not limited to, once daily and can also be twice a day (b.i.d.).
In a preferred embodiment, when the disorder is osteoarthritis (e.g., osteoarthritis of the knee, hip, spine and shoulder) or refractory osteoarthritis, lumiracoxib is to be administered at a dose of about 100 mg once daily, about 200 mg once daily or about 400 mg once daily.
In another embodiment, when the disorder is dysmenorrhea, lumiracoxib is to be administered at a dose of about 200 mg once daily or 400 mg once daily.
In still another embodiment, when the disorder is acute gout, lumiracoxib is to be administered at a the dose of about 200 mg once daily or 400 mg once daily.
In a further embodiment, when the disorder is resulting in acute pain, the dose to be administered is about 400 mg once daily.
The following aspects are also included in the present disclosure:

Lumiracoxib for use in treating a cyclooxygenase-2 dependent disorder in a patient, wherein the patient is selected on the basis of genetic polymorphisms in HLA genes present in the patient, and wherein the genetic polymorphisms are indicative of the presence or prediction of hepatotoxicity of lumiracoxib, and wherein lumiracoxib is to be administered to a patient being non carrier of said polymorphisms.

The use of lumiracoxib in the manufacture of a medicament for the treatment of a cyclooxygenase-2 dependent disorder in a patient, wherein the patient is selected on the basis of genetic polymorphisms in HLA genes present in the patient, wherein the genetic polymorphisms are indicative of the presence or prediction of hepatotoxicity of lumiracoxib, and wherein lumiracoxib is to be administered to a patient being non carrier of said polymorphisms.

The genetic polymorphisms or the HLA alleles are preferably selected from the group consisting of one or more of DQA1*0102, DRB1*1501, DQB1*0602 and DRB5*0101. Lumiracoxib for use in treating a cyclooxygenase-2 dependent disorder in a patient that has a reduced predisposition or risk of developing hepatotoxicity in response to lumiracoxib, wherein said reduced predisposition or risk is identified by the method set forth in any of claims 1-14.

The use of lumiracoxib for the manufacture of a medicament for treating a cyclooxygenase-2 dependent disorder in a patient that has a reduced predisposition or risk of developing hepatotoxicity in response to lumiracoxib, wherein said reduced predisposition or risk is identified by the method set forth in any of claims 1-11.

Lumiracoxib for use in the treatment of a cyclooxygenase-2 disorder in a patient, wherein the patient is not a carrier of one or more of HLA allele(s) selected from the group consisting of DQA1*0102, DRB1*1501, DQB1*0602 and DRB5*0101.

Use of lumiracoxib for the manufacture of a medicament for treating a cyclooxygenase-2 dependent disorder in a patient that has a reduced predisposition or risk of developing hepatotoxicity in response to lumiracoxib, wherein said reduced predisposition or risk is identified by the method set forth in any of claims 1-11.

Use of lumiracoxib in the manufacture of a medicament for the treatment of a cyclooxygenase-2 mediated disorder in a patient, wherein the patient is not a carrier of one or more of HLA allele(s) selected from the group consisting of DQA1*0102, DRB1*1501, DQB1*0602 and DRB5*0101.

Preferably the HLA allele is DQA1*0102.
Preferably, the patient or subject is preferably human.
In a still further embodiment, liver function monitoring is preferably to be performed at baseline and monthly thereafter.
All patients should have baseline liver function tests prior to commencing treatment. Patients with transaminases >1.5xULN should not commence therapy with lumiracoxib.
If treatment for more than 30 days is required liver function tests should be repeated at monthly intervals (see actions to be taken below) and patients reviewed before continued prescription. If AST/ALT levels >5xULN develop, lumiracoxib therapy should be discontinued. If AST/ALT levels >3xULN are detected, then lumiracoxib may be continued, but the liver function tests should be repeated in 7 days. If AST/ALT levels >3xULN persist upon retesting, lumiracoxib should be withdrawn.
Lumiracoxib should be discontinued if clinical or laboratory signs and/or symptoms consistent with liver disease develop (e.g., jaundice).
Alternatively, the present methods of prediction, and methods of treatment and uses also apply to naproxen as a COX-2 inhibitor.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The following Examples are presented in order to more fully illustrate the preferred embodiments of the disclosure. These examples should in no way be construed as limiting the scope of the disclosed subject matter, as defined by the appended claims.

### Example 1

### Exploratory Whole Genome Association Analysis

An initial case-control exploratory whole genome association study is conducted using 41 lumiracoxib-treated patients with ALT/AST>5xULN, matched to 176 controls on the basis of clinical study, sex, race, age ±2 years (where possible), and country (where possible). Genomic DNA samples are obtained from the 41 affected patients and 176 control patients. PCR is performed using the GenomiPhi V2 DNA Amplification Kit (GE Healthcare, Piscataway, N.J.) with general protocol publication 2-6600-30WP Rev B 2006 and carried out on a Mycycler (BioRad, Hercules, Calif.). The quantification protocol is modified such that the dilution of the GenomiPhi V2 amplification products is 1:5 instead of 1:10. After fluorescence is measured using the SpectraMax M2 plate reader (Molecular Devices, Sunnyvale, Calif.), microarray genotyping is performed using the Genome-Wide SNP Array 6.0 kit (Affymetrix, Santa Clara, Calif.). Chips with an initial call rate greater than 84% are genotyped with the Birdseed algorithm in apt-probeset-genotype (Affymetrix, Santa Clara, Calif.).

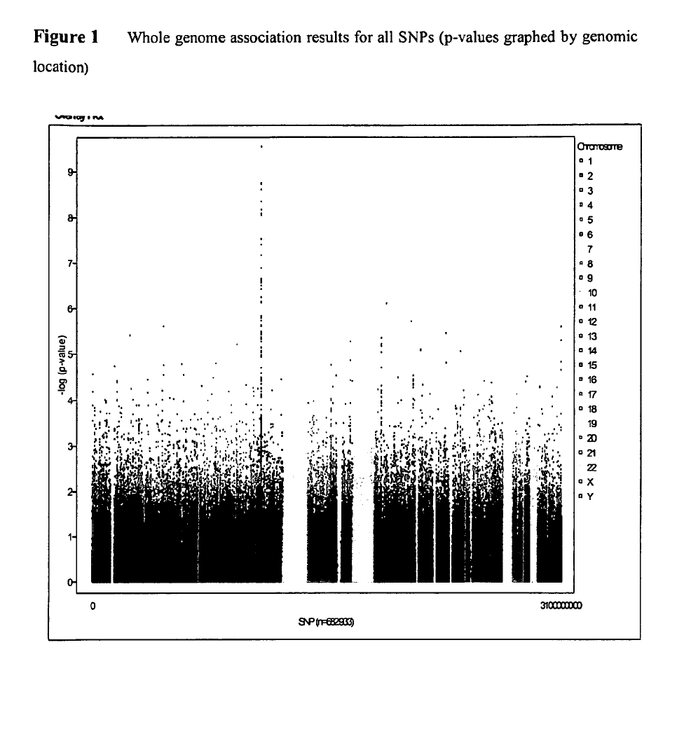

The results for the analysis are shown in FIG. 1. A large peak of associated SNPs is observed on chromosome 6 with rs9270986 yielding the most significant result) (p=2.8×10⁻¹⁰). The majority of the SNPs under this peak are located in the extended MHC region with the most significant findings mapping to the MHC class II region. A total of 7 SNPs are still statistically significant (p<0.05) after correcting for multiple comparisons (Table 1) with rs9270986 yielding the most significant study-wide finding (p=0.0075).

TABLE 1
Significant findings from the exploratory whole genome
association study after multiple testing corrections

| | | | Nominal | Study-wide |
|---|---|---|---|---|
| rs number | Chromosome | Position | p-value | p-value |
| rs9270986 | 6 | 32682038 | 2.8 × 10⁻¹⁰ | 0.0075 |
| rs3129900 | 6 | 32413957 | 1.8 × 10⁻⁹ | 0.022 |
| rs3132943 | 6 | 32416443 | 1.9 × 10⁻⁹ | 0.023 |
| rs3129934 | 6 | 32444165 | 2.5 × 10⁻⁹ | 0.026 |
| rs3135365 | 6 | 32497233 | 4.5 × 10⁻⁹ | 0.038 |
| rs3129932 | 6 | 32444105 | 6.5 × 10⁻⁹ | 0.047 |
| rs910049 | 6 | 32423705 | 6.6 × 10⁻⁹ | 0.047 |

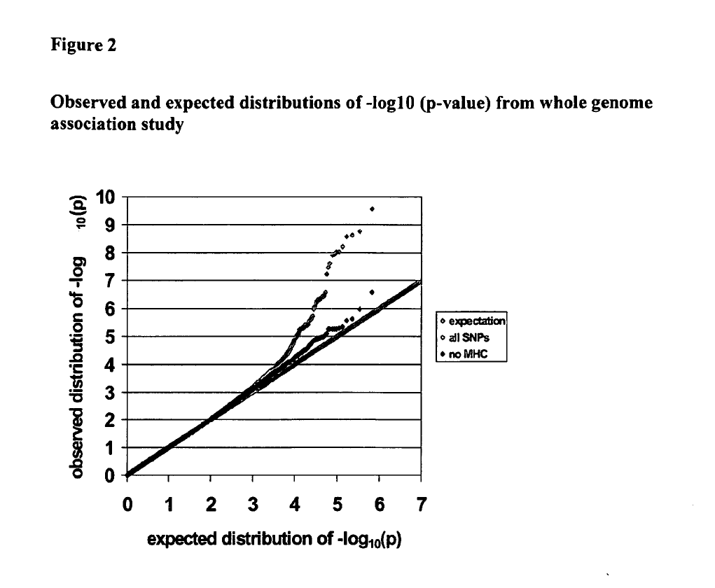

A comparison of the observed and the expected distribution of p-values (FIG. 2) show that most of the significant findings from the whole genome analysis occur in the MHC region.

### Example 2

### Replication Study in an Independent Set of Cases with Elevated Liver Enzymes (>3xULN ALT and/or AST)

The replication study is performed on the most significant SNPs in the genome-wide scan. This analysis is performed on an independent set of 98 TARGET cases and 405 controls matched to the cases on a roughly 4:1 basis using the matching criteria described previously. These 98 cases comprise all of the remaining available TARGET lumiracoxib recipients with >3xULN ALT/AST. The results of the replication study for the top two SNPs from the whole-genome association study are shown in table 2.

TABLE 2
Replication study results
(>3xULN ALT and/or AST)

| | | | | | | Carrier | Carrier |
|---|---|---|---|---|---|---|---|
| | | HWE | | | MAF | case | control |
| rs | Gene/ | p- | p- | MAF | con- | fre- | fre- |
| number | region | value | value | cases | trols | quency | quency |
| 9270986 | MHC | 0.17 | 1.0 × 10⁻⁹ | 34.5% | 14.8% | 59.8% | 26.9% |
| 3129900 | MHC | 0.71 | 4.4 × 10⁻¹² | 36.7% | 14.8% | 61.2% | 27.3% |

HWE = Hardy-Weinberg Equilibrium,
MAF = Minor Allele Frequency

### Example 3

### Genetic Fine Mapping and Detection of Associated HLA Alleles

A genetic fine mapping study is undertaken in order to identify potentially causative polymorphisms in the MHC class II region. The genotyping of HLA alleles is performed for HLA-DRB1, HLA-DRB3-5, HLA-DQA1, and HLA-DQB1. A total of 139 patients with >3xULN ALT/AST and 581 matched controls are used for a case-control analysis. The 139 cases with >3xULN ALT/AST are the sum total of all the cases used for the previous analyses described in the whole genome association analysis and the replication analysis. They account for all the cases that are available for pharmacogenetic analysis from the TARGET study. Two cases failed genotyping for the HLA alleles, leaving 137 cases available for the HLA analysis. Of these, 76 have ULN values >3x but ≦5x, and 61 have ULN values >5x. The 581 controls are also the sum total of all the controls used for the previous analyses. Four of these failed genotyping for the HLA alleles, leaving 577 controls available for the HLA analysis. The most significant findings are shown in Table 3. Four alleles yielded highly significant associations, with the HLA-DRB1*1501 allele having the most significant association (p=6.8×10⁻²⁵). This set of genes and alleles is part of a very well-characterized haplotype (DRB1*1501-DQB1*0602-DRB5*0101-DQA1*0102).

TABLE 3
Most significant HLA genes and alleles associated with elevated
liver enzymes (>3xULN ALT/AST): (137 cases and 577 controls)

| Gene/allele | p-value |
|---|---|
| DRB1*1501 | 6.8 × 10⁻²⁵ |
| DQB1*0602 | 1.1 × 10⁻²² |
| DRB5*0101 | 1.6 × 10⁻²⁰ |
| DQA1*0102 | 1.2 × 10⁻¹⁸ |

### 3.1—Detection of HLA Allele DQA1*0102

Genomic DNA samples are obtained from 137 patients having elevated liver enzymes greater than or equal to 3 times the upper level of normal (ULN) after treatment with lumiracoxib and 577 matched controls. Genomic DNAs are extracted from each patient's blood using Gentra systems PUREGENE D-50K DNA Isolation Kit (Qiagen, CA).

Low resolution genotyping of the extracted genomic DNA is performed using the LABType® Sequence-Specific Oligonucleotide DQA1/DQB1 Typing Test Kit (One Lambda, Inc., Canoga Park, Calif.) having Lot No. #003 coupled with the Luminex xMAP® technology, according to the manufacturer's instructions. Additional genotype testing is performed on any remaining non distinguished genomic DNA using the Olerup Sequence Specific Primer DQA1 Test Kit (GenoVision, Inc., West Chester, Pa.) having Lot Y46.

Based upon analysis of these genomic DNA samples, the study indicates that the sensitivity and specificity for HLA allele DQA1*0102 are 73.7% and 69.2%, respectively, for >3xULN ALT and/or AST.

### 3.2—Detection of HLA Allele DRB1*1501

Genomic DNA samples are obtained from 137 patients having elevated liver enzymes greater than or equal to 3 times the upper level of normal after treatment with lumiracoxib, and 577 matched controls. Genomic DNAs are extracted from each patient's blood using Gentra systems PUREGENE D-50K DNA Isolation Kit (Qiagen, CA). High resolution genotyping of the extracted genomic DNA is performed using the LABType® Sequence-Specific Oligonucleotide DRB1 High Definition Typing Test Kit, Lot #002 (One Lambda, Inc., Canoga Park, Calif.). Based upon analysis of these genomic DNA samples, the study indicates that the sensitivity and specificity for HLA allele DRB1*1501 are 64.2% and 80.8%, respectively, for >3xULN ALT and/or AST.

### 3.3—Detection of HLA Allele DQB1*0602

Genomic DNA samples are obtained from 137 patients having elevated liver enzymes greater than or equal to 3 times the upper level of normal after treatment with lumiracoxib, and 577 matched controls. Genomic DNAs are extracted from each patient's blood using Gentra systems PUREGENE D-50K DNA Isolation Kit (Qiagen, CA). HLA-DQB1 genotyping is performed using the LABType SSO DQA1/DQB1 Typing test kit, lot #003 (One Lambda, Inc) coupled with the Luminex xMAP® technology, according to the manufacturer's instructions. Further ambiguities are resolved using Olerup SSP™ kits DQB1 03, 04, 05, 06 Lot V55, K42, X15, V26 from Genovision. DQB1*06 ambiguities are resolved using the Olerup SSP™ DQB1*06, Lot V26 from Genovision (Qiagen). Genovision Helmberg SCORE software is used to assign allele designations. Based upon analysis of these genomic DNA samples, the study indicates that the sensitivity and specificity for HLA allele DQB1*0602 are 62.0% and 80.8%, respectively, for >3xULN ALT and/or AST.

### 3.4—Detection of HLA Allele DRB5*0101

Genomic DNA samples are obtained from 137 patients having elevated liver enzymes greater than or equal to 3 times the upper level of normal after treatment with lumiracoxib, and 577 matched controls. Genomic DNAs are extracted from each patient's blood using Gentra Systems PUREGENE D-50K DNA Isolation Kit (Qiagen, CA). HLA-DRB3, 4, 5 genotyping is performed using the LABType® SSO DRB3, 4, 5, Typing test, lot #007 (One Lambda, Inc) coupled with the Luminex xMAP® technology, according to the manufacturer's instructions. Ambiguities are further resolved using Olerup SSP™ kits DRB3*, B4*, B5*, Lot Y16, Y01, X51. In rare cases additional ambiguities are resolved using IMGM in-house sequence-based-typing (SBT) test. High resolution genotyping to identify allele DRB5*0101 is pursued using the Olerup SSP™ DRB5, Lot X51 from Genovision (Qiagen, CA) according to the manufacturer's instructions. Genovision Helmberg SCORE software is used to assign allele designations. Sequencing to resolve ambiguities is used according to the IHWG technical manual (International Histocompatibility Working Group). Based upon analysis of these genomic DNA samples, the study indicates that the sensitivity and specificity for HLA allele DRB5*0101 are 64.2% and 80.1%, respectively, for >3xULN ALT and/or AST.

### 3.5—Correlation Between a Patient's Risk of Developing Hepatotoxicity and HLA Alleles DQA1*0102, DRB1*1501, DQB1*0602 and DRB5*0101

Affected patients and matched controls are identified from a large 52-week international, multicenter, stratified, randomized, double-blind, double-dummy, parallel group clinical study. Most patients are administered one 400 mg dose of lumiracoxib each day. Patients are identified as affected cases based upon clinical measurements of elevated liver enzymes greater than or equal to 3 times ULN following administration of lumiracoxib. Patients are identified as control cases based upon clinical measurements of elevated liver enzymes less than 3 times ULN following administration of lumiracoxib, and control patients are matched to affected cases based upon clinical trial, country (where possible), sex, race and age (within 2 years, where possible). A total of 137 cases and 577 matched controls are used for analysis.

Genomic DNA samples obtained from the 137 affected patients and 577 control patients are used for the HLA analysis. Genomic DNA is extracted and genotyped as described above. Each of HLA alleles DQA1*0102, DRB1*1501, DQB1*0602 and DRB5*0101 are independently analyzed within each genomic DNA sample.

The HLA allele DQA1*0102 is demonstrated to have a strong association with hepatotoxicity in patients treated with lumiracoxib and having a p-value of 1.2×10⁻¹⁸. The allele frequency of DQA1*0102 is 42.7% for affected patients and 17.4% for control patients. The sensitivity, specificity, positive predictive value and negative predictive value for HLA allele DQA1*0102 associated with the development of hepatotoxicity in response to treatment with lumiracoxib are 73.7%, 69.2%, 6.1% and 98.98%, respectively, with a relative risk of 6.0 for the study. With such a sensitivity and high negative predictive value, typing of the HLA allele DQA1*0102 can be used in identifying high-risk patients for lumiracoxib-induced hepatotoxicity.

The HLA allele DRB1*1501 is demonstrated to have a strong association with hepatotoxicity in patients treated with lumiracoxib and having a p-value of 6.8×10⁻²⁵. The allele frequency of DRB1*1501 is 35.4% for affected patients and 10.5% for control patients. The sensitivity, specificity, positive predictive value and negative predictive value for HLA allele DRB1*1501 associated with the development of hepatotoxicity in response to treatment with lumiracoxib are 64.2%, 80.8%, 8.3% and 98.82%, respectively, with a relative risk of 7.0 for the study. With such a sensitivity and high negative predictive value, typing of the HLA allele DRB1*1501 can be used in identifying high-risk patients for lumiracoxib-induced hepatotoxicity.

The HLA allele DQB1*0602 is demonstrated to have a strong association with hepatotoxicity in patients treated with lumiracoxib and having a p-value of 1.1×10⁻²². The allele frequency of DQB1*0602 is 34.3% for affected patients and 10.5% for control patients. The sensitivity, specificity, positive predictive value and negative predictive value for HLA allele DQB1*0602 associated with the development of hepatotoxicity in response to treatment with lumiracoxib are 62.0%, 80.8, 8.0% and 98.74%, respectively, with a relative risk of 6.4 for the study. With such a high negative predictive value, typing of the HLA allele DQB1*0602 can be used in identifying high-risk patients for lumiracoxib-induced hepatotoxicity.

The HLA allele DRB5*0101 is demonstrated to have a strong association with hepatotoxicity in patients treated with lumiracoxib and having a p-value of 1.6×10⁻²⁰. The allele frequency of DRB5*0101 is 32.1% for affected patients and 10.0% for control patients. The sensitivity, specificity, positive predictive value and negative predictive value for HLA allele DRB5*0101 associated with the development of hepatotoxicity in response to treatment with lumiracoxib are 64.2%, 80.1%, 8.0% and 98.81%, respectively, with a relative risk of 6.7 for the study. With such a sensitivity and high negative predictive value, typing of the HLA allele DRB5*0101 can be used in identifying high-risk patients for lumiracoxib-induced hepatotoxicity.

### 3.6—Genetic Markers Equivalent to HLA DQA1*0102, DRB1*1501, DQB1*0602, and DRB5*0101

To test the equivalent genetic markers to HLA alleles DQA1*0102 and DRB1*1501, the single nucleotide polymorphism identified as rs9270986 (by the NCBI Database) is analyzed for its association with patient development of hepatotoxicity in response to lumiracoxib in 134 cases with ALT and/or AST>3xULN and 566 matched controls. These represent all patients who are successfully genotyped using the Affymetrix 6.0 array. rs9270986 is a single nucleotide polymorphism located between the HLA-DRB1 and HLA-DQA1 genes at position 32682038 on Chromosome 6 (position 23432310 on accession NT_{—}007592).

The SNP rs9270986 is demonstrated to have a strong association with hepatotoxicity in patients treated with lumiracoxib and having a p-value of 3.6×10⁻¹⁸. The minor allele frequency of the marker is 37.3% for affected patients and 13.8% for control patients. The sensitivity, specificity, positive predictive value and negative predictive value for the SNP rs9270986 associated with the development of hepatotoxicity in response to treatment with lumiracoxib are 66.4%, 75.0%, 6.7%, 98.80% respectively, with a relative risk of 5.6 for the study. With such a sensitivity and high negative predictive value, the detection of SNP rs9270986 can be used in identifying high-risk patients for lumiracoxib-induced hepatotoxicity.

3.7—DQA1*0102 Marker Performance in Patients with Increasing ULN Thresholds
Table 4 compares the percentage of cases carrying the DQA1*0102 allele (i.e., sensitivity) across increasing thresholds of ULN ALT/AST. The sensitivity for DQA1*0102 allele is 73.7% for the >3xULN patients and improves as the threshold of ULN increases, reaching 100% for the >20x cases (8 of 8 carry the DQA1*0102 allele). This data indicates that the marker's performance improves as the severity of the liver enzyme elevation increases.

TABLE 4
Sensitivity and number of cases with the DQA1*0102 allele

| | | Number of cases with | Total number of |
|---|---|---|---|
| ULN ALT/AST | Sensitivity | DQA1*0102 allele | genotyped cases |
| >3x | 73.7% | 101 | 137 |
| >5x | 83.6% | 51 | 61 |
| >8x | 90.9% | 30 | 33 |
| >10x | 91.7% | 22 | 24 |
| >15x | 93.8% | 15 | 16 |
| >20x | 100% | 8 | 8 |

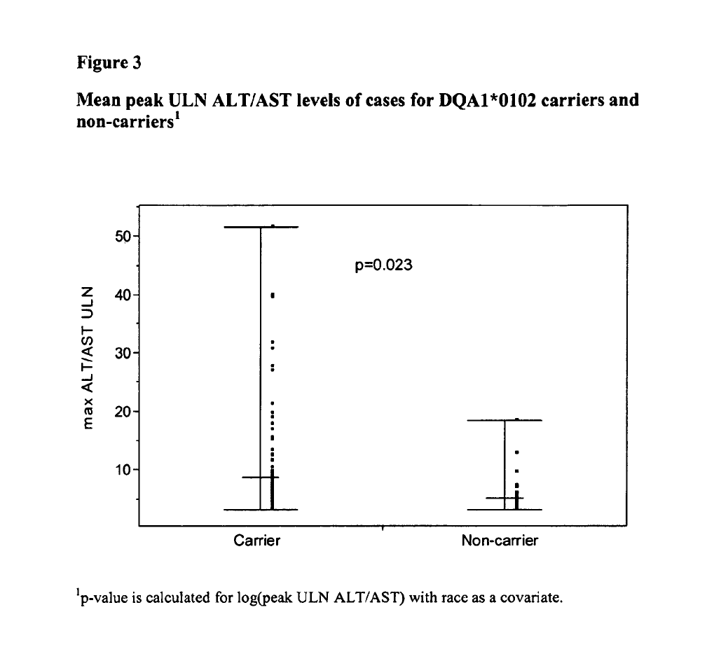

Also, if the mean peak levels of ULN ALT/AST are compared, the DQA1*0102 non-carriers have lower mean peak ULN ALT/AST value compared to the carriers (5.1 non-carriers vs. 8.6 carriers) [p=0.023 for comparison of mean log(peak ULN ALT/AST), adjusted for race as a covariate] (FIG. 3). Taken together these results demonstrate that the most severe cases are more strongly correlated with allele carrier status.
Consistent with this are the findings for the Hy's law cases. There are three Hy's law cases in the TARGET study for which DNA is available for analysis. All three of these cases are heterozygous for the DRB1*1501, DRB5*0101 and DQA1*0102 alleles (Table 5)

TABLE 5
HLA genotypes for Hy's law cases (n = 3)

| Hy's law case | DRB1*1501 | DQA1*0102 | DRB5*0101 | DQB1*0602 |
|---|---|---|---|---|
| Patient 1 | Heterozygous | Heterozygous | Heterozygous | Heterozygous |
| Patient 2 | Heterozygous | Heterozygous | Heterozygous | Heterozygous |
| Patient 3 | Heterozygous | Heterozygous | Heterozygous | Non-carrier |
| | | | | (carrier for |
| | | | | *0601 allele) |

Two of the three cases are heterozygous for the DQB1*0602 allele while the third is a carrier for the closely related DQB1*0601 allele.
A further analysis is undertaken to determine if there is a difference between DQA1*0102 carriers (homozygotes and heterozygotes) and non-carriers in the types of liver injury. This data is shown in Table 6. The data for the >3xULN ALT/AST patients indicates that there is a difference between carriers and non-carriers with regard to the type of liver injury. A total of 76.0% of the DQA1*0102 carriers have hepatocellular liver injury and 21.0% have mixed liver injury, while 47.2% of the DQA1*0102 non-carriers have hepatocellular liver injury and 47.2% have mixed liver injury (p=0.0015). These data strongly suggest that the type of liver injury is different between DQA1*0102 carriers and non-carriers

TABLE 6
Type of liver injury by DQA1*0102 carrier
status and ULN ALT/AST thresholds
>3xULN ALT/AST

| Type of liver injury | Carrier | Non-carrier |
|---|---|---|
| Hepatocellular | 76 (76.0%) | 17 (47.2%) |
| Mixed | 21 (21.0%) | 17 (47.2%) |
| Cholestatic | 3 (3.0%) | 2 (5.6%) |
| total | 100 | 36 |
| | p = 0.0015 | |

### 3.8—Liver Enzymes (ALST/AST) Elevation Rates in TARGET Study:

The ULN ALT/AST rates for lumiracoxib, ibuprofen, naproxen and lumiracoxib DQA1*0102-negative patients from the TARGET study are shown in Table 7. Compared to the lumiracoxib arm, the rates for lumiracoxib DQA1*0102-negative patients are reduced considerably and are comparable to the other NSAIDs. It is worth noting that the naproxen and ibuprofen arms each had 2 Hy's law cases, while the DQA1*0102-negative patients in the lumiracoxib arm had no cases (3 of 3 Hy's law cases with DNA carried the DQA1*0102 allele). Nine Hy's law cases are reported in the lumiracoxib arms of TARGET but only 3 are used in the pharmacogenetic analysis. No DNA or informed consent is obtained for 5 of the remaining cases, while the 6^{th} case is not successfully genotyped.

TABLE 7
Liver enzyme (ALT/AST) elevation crude rates in TARGET

| | | | | | ALT/AST >3x |
|---|---|---|---|---|---|
| | | ALT/AST >3x | ALT/AST >5x | ALT/AST >8x | ULN + bili- |
| | | ULN | ULN | ULN | rubin ≧2xULN |
| Treatment group | N | n (%) | n (%) | n (%) | n (%) |
| Lumiracoxib | 8961 | 236 (2.6) | 113 (1.3) | 56 (0.62) | 9 (0.10) |
| Lumiracoxib (with | ~3538¹ | 36 (1.0) | 10 (0.3) | 3 (0.1) | 0 (0) |
| DNA and excluding | | | | | |
| DQA1*0102 patients) | | | | | |
| Ibuprofen | 4309 | 35 (0.8) | 13 (0.3) | 4 (0.1) | 2 (0.05) |
| Naproxen | 4630 | 21 (0.5) | 6 (0.1) | 3 (0.1) | 2 (0.04) |

¹All the cases (n = 137) with available DNA are genotyped for this analysis but only a subset of controls (n = 577) are genotyped. The total number of DQA1*0102-negative patients with DNA is estimated by assuming that the DQA1*0102 carrier frequency among non-genotyped controls is equal to that among genotyped controls, and that the frequency of elevated liver enzymes (>3x ULN ALT/AST) among the non-DNA/consent population is equal to that among the DNA/consent population.

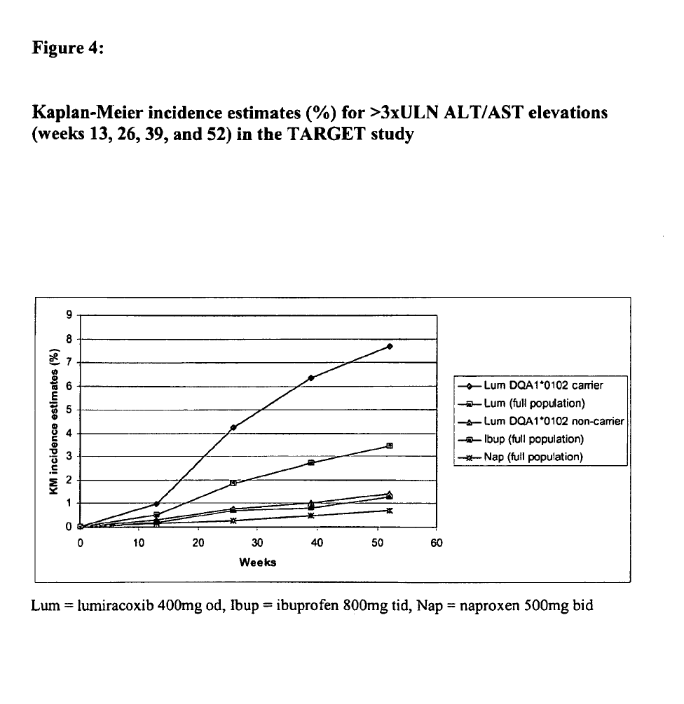

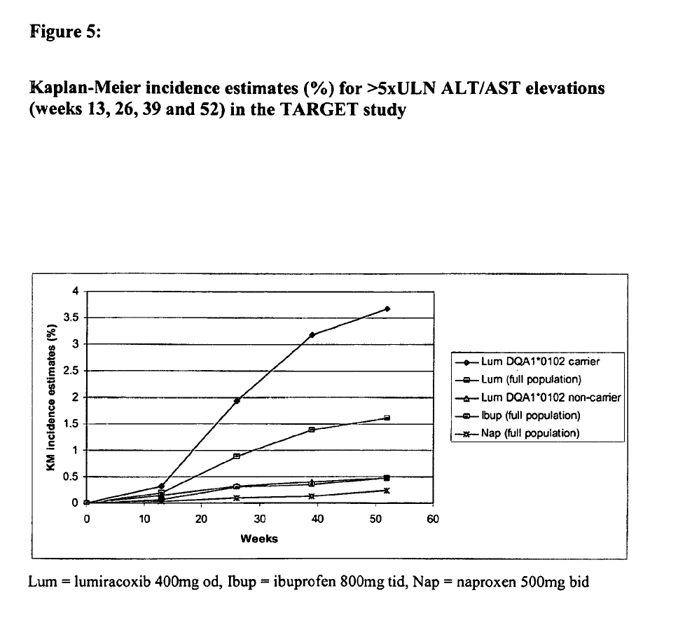

Kaplan-Meier incidence estimates are calculated for the DQA1*0102 carriers and non-carriers for >3xULN (FIG. 4) and >5xULN (FIG. 5) ALT/AST elevations. The DQA1*0102 carriers show a dramatic increase in the incidence after week 13, which suggests that the autoimmune based liver toxicity does not manifest clinically until after several weeks of exposure to lumiracoxib. This is in contrast to the non-carriers which do not show a sharp increase after week 13, and show a consistent and more gradual slope throughout the study. The non-carriers have an incidence remarkably similar to the ibuprofen patients as well. These differences in the Kaplan-Meier incidence estimates suggest that the mechanism of hepatotoxicity is different between DQA1*0102 carriers and non-carriers.

### Example 4

### Further Exploratory Genome-Wide Analysis Using all TARGET Cases with >3xULN ALT/AST

The genome-wide analysis was repeated using all the samples >3xULN ALT/AST cases. Of the 139 cases and 581 controls initially available, 5 cases and 15 controls failed genotyping on the Affymetrix microarray or failed HLA genotyping, leaving 134 cases and 566 controls available for the analysis. The results for the top 5 most significant SNPs are shown in Table 8. Similar to the initial genome-wide analysis, all the top SNPs are in the MHC region.

TABLE 8
Top 5 results for exploratory genome-wide analysis
of >3xULN ALT/AST cases for entire dataset

| rs number | Chromosome | Position | p-value |
|---|---|---|---|
| rs3131294 | 6 | 32288124 | 4.0 × 10⁻²¹ |
| rs3129868 | 6 | 32512355 | 1.5 × 10⁻¹⁸ |
| rs9270986 | 6 | 32682038 | 3.6 × 10⁻¹⁸ |
| rs3129900 | 6 | 32413957 | 8.3 × 10⁻¹⁸ |
| rs3135365 | 6 | 32497233 | 6.0 × 10⁻¹⁷ |

The study described here offers clear and strong evidence for an association between polymorphisms in the MHC class II region and lumiracoxib-induced hepatotoxicity. Starting with an exploratory whole genome association study, followed by the replication in an independent set of cases and controls, and finally concluding with the identification of associated HLA alleles, this study produced an association result (6.8×10⁻²⁵) that achieves an extremely high level of statistical confidence.

1-48. (canceled)

49. A method of identifying or predicting the predisposition to hepatotoxicity or the risk of developing hepatotoxicity and/or elevated ALT or AST in a human subject treated with lumiracoxib comprising assaying a biological sample obtained from a subject for the presence of at least one HLA allele selected from the group consisting of DQA1*0102, DRB1*1501, DQB1*0602 and DRB5*0101, wherein the presence of said at least one HLA allele is indicative of the presence or increased prediction of hepatotoxicity and/or elevated ALT or AST or an increased risk of developing hepatotoxicity in said subject, and wherein the absence of said at least one HLA allele is indicative of the absence or decreased prediction of hepatotoxicity or a decreased risk of developing hepatotoxicity in said subject.

50. The method according to claim 49, wherein the at least one HLA allele is DQA1*0102.

51. The method according to claim 49, wherein said biological sample is selected from the group consisting of blood, blood-derived product, lymph, urine, tear, saliva, cerebrospinal fluid, buccal swabs, sputum, leukocyte sample or tissue samples or any combination thereof.

52. The method for predicting Hy's law cases (>3xULN ALT/AST and ≧2xULN serum bilirubin) upon administration of lumiracoxib, comprising assaying a biological sample obtained from a subject for the presence of at least one HLA allele selected from the group of DQA1*0102, DRB1*1501, DRB5*0101 and DQB1*0602 in said subject.

53. The method according to claim 49, wherein the presence of said HLA allele is determined by using at least one oligonucleotide that specifically hybridizes with the nucleic acid coding for the allele.

54. The method according to claim 49, wherein the presence of said HLA alleles is detected by Sanger-based sequencing, direct sequencing, new generation sequencing or next generation sequencing.

55. The method according to claim 49, wherein the presence of said HLA allele is detected by sequence-specific primer (SSP) typing, sequence-specific oligonucleotide (SSO) typing, sequence based typing (SBT), DNA amplification, microarray analysis, northern blot analysis, or reverse transcription PCR.

56. The method according to claim 55, wherein sequencing is performed subsequently to sequence-specific oligonucleotide (SSO) typing, sequence-specific primer (SSP) typing, DNA amplification such as polymerase chain reaction (PCR), microarray analysis, northern blot analysis, or reverse transcription PCR.

57. The method according to claim 49, wherein the presence of said HLA allele is detected by using a hybridization assay.

58. A method of treating a cyclooxygenase-2 dependent disorder by administering lumiracoxib to a subject that has a reduced predisposition or risk of developing hepatoxicity in response to lumiracoxib, wherein said reduced predisposition or risk is identified by a method set forth in claim 49.

59. A method of treating a cyclooxygenase-2 dependent disorder in a human subject comprising the steps of:
(i) receiving data regarding the presence in a biological sample obtained from said subject of at least an HLA allele selected from the group consisting of DQA1*0102, DRB1*1501, DQB1*0602 and DRB5*0101, said HLA allele being indicative of the presence or prediction of hepatotoxicity,
(ii) administering lumiracoxib to the subject if said received data indicates that the subject is not a carrier of said HLA allele.

60. The method according to claim 59, wherein the biological sample is selected from the group consisting of blood, blood-derived product, lymph, urine, tear, saliva, cerebrospinal fluid, buccal swabs, sputum, leukocyte sample or tissue samples or any combination thereof.

61. The method according to claim 49, wherein said HLA allele is DQA1*0102.

62. The method according to claim 59, wherein the cycloxygenase-2 dependent disorder is selected from an inflammatory disorder, osteoarthritis, rheumatoid arthritis, refractory osteoarthritis, ankylosing spondylitis, gout, dental pain, post-surgery dental pain, post-operative pain, orthopaedic surgery pain, low back pain, sore throat, post-herpetic neuralgia, herpes zoster, trigeminal neuralgia, visceral pain, musculoskeletal pain, fibromyalgia, dysmenorrhea, renal and biliary colic, migraine, headache, pain associated with cancer, pyresis, a neurodegenerative disease, Alzheimer's disease, osteoporosis, asthma, lupus, psoriasis, neoplasia, and angiogenesis-mediated ocular diseases.

63. The method according to claim 59, wherein the cycloxygenase-2 dependent disorder is selected from the group consisting of osteoarthritis, rheumatoid arthritis, refractory osteoarthritis, ankylosing spondylitis, gout, low back pain, dental pain, post-surgery dental pain, visceral pain, musculoskeletal pain, post-herpetic neuralgia, herpes zoster, trigeminal neuralgia, fibromyalgia, and dysmenorrhoea.

64. The method according to claim 59, wherein the step of administering comprises administering from about 25 mg to about 1200 mg lumiracoxib.

65. The method according to claim 59, wherein the step of administering comprises administering from about 100 mg to about 400 mg lumiracoxib.

66. The method according to claim 59, wherein the disorder is osteoarthritis or refractory osteoarthritis, and wherein the step of administering comprises administering lumiracoxib in a dose of about 100 mg once daily, about 200 mg once daily or about 400 mg once daily.

67. The method according to claim 59, wherein the disorder is dysmenorrhea, and wherein the step of administering comprises administering lumiracoxib in a dose of about 200 mg once daily or 400 mg once daily.

68. The method according to claim 59, wherein the disorder is acute gout, and wherein the step of administering comprises administering lumiracoxib in a dose of about 200 mg once daily or 400 mg once daily.

69. The method according to claim 59, wherein the disorder results in acute pain, and wherein the step of administering comprises administering lumiracoxib in a dose of about 400 mg once daily.

70. A kit comprising at least one probe for detecting a region of HLA allele DQA1*0102 and lumiracoxib.


SEQUENCE LISTING (18 sequences)

SEQ ID NO: 1 - DNA, 768 bp, homo sapiens
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 2 - DNA, 768 bp, homo sapiens
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatctgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 3 - DNA, 768 bp, homo sapiens
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacctggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 4 - DNA, 768 bp, homo sapiens
atgatcctaaacaaagctctgctgctgggggccctcgctctgaccactgtgatgagcccctgtggaggtgaagacattgtggctgaccacgttgcctcttgtggtgtaaacttgtaccagttttacggtccctctggccagtacacccatgaatttgatggagatgagcagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatctgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgtgccctggggttgtctgtgggcctcatgggcattgtggtgggcactgtcttcatcatccaaggcctgcgttcagttggtgcttccagacaccaagggccattgtga

SEQ ID NO: 5 - protein, 254 aa, homo sapiens
MILNKALLLGALALTTVMSPCGGEDIVADHVASCGVNLYQFYGPSGQYTHEFDGDEQFYVDLERKEYAWRWPEFSKFGGFDPQGALRNMAVAKHNLNIMIKRYNSTAATNEVPEVTVFSKSPVTLGQPNTLICLVDNIFPPVVNITWLSNGQSVTEGVSETSFLSKSDHSFFKISYLTFLPSADEIYDCKVEHWGLDQPLLKHEPEIPAPMSELTETVVCALGLSVGLMGIVVGTVFIIQGLRSVGASRHQGPL

SEQ ID NO: 6 - DNA, 801 bp, homo sapiens
atggtgtgtctgaagctccctggaggctcctgcatgacagcgctgacagtgacactgatggtgctgagctccccactggctttgtctggggacacccgaccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtccaacctaaggtgactgtatatccttcaaagacccagcccctgcagcaccacaacctcctggtctgctctgtgagtggtttctatccaggcagcattgaagtcaggtggttcctgaacggccaggaagagaaggctgggatggtgtccacaggcctgatccagaatggagactggaccttccagaccctggtgatgctggaaacagttcctcgaagtggagaggtttacacctgccaagtggagcacccaagcgtgacaagccctctcacagtggaatggagagcacggtctgaatctgcacagagcaagatgctgagtggagtcgggggctttgtgctgggcctgctcttccttggggccgggctgttcatctacttcaggaatcagaaaggacactctggacttcagccaacaggattcctgagctga

SEQ ID NO: 7 - DNA, 266 bp, homo sapiens
cacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggagttgtggagagcttcacagtgcagcgg

SEQ ID NO: 8 - DNA, 266 bp, homo sapiens
cacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctattgcagacacaactacggggttgtggagagcttcacagtgcagcgg

SEQ ID NO: 9 - DNA, 270 bp, homo sapiens
cacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgatgccgagtactggaacagccagaaggacatcctggagcaggcgcgggccgcggtggacacctactgcagacacaactacggggttgtggagagcttcacagtgcagcggcgag

SEQ ID NO: 10 - DNA, 270 bp, homo sapiens
cacgtttcctgtggcagcctaagagggagtgtcatttcttcaatgggaccgagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgag

SEQ ID NO: 11 - DNA, 270 bp, homo sapiens
cacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgaatactggaacagccagaaggacatcctggagcaggcgcgggccgcggtggacacctactgcagacacaactacggggttgtggagagcttcacagtgcagcggcgag

SEQ ID NO: 12 - protein, 263 aa, homo sapiens
MVCLKLPGGSCMTALTVTLMVLSSPLALSGDTRPRFLWQPKRECHFFNGTERVRFLDRYFYNQEESVRFDSDVGEFRAVTELGRPDAEWNSQKDILEQARAAVDTYCRHNYGVVESFTVQRRVQPKVTVYPSKTQPLQHHNLLVSVSGFYPGSIEVRWFLNGQEEKAGMVSTGLIQNGDWTFQTLVMLETVPRSGEVYTCQVEHPSVTSPTVEWRARSESAQSKMLSGVGGFVLGLLFLGAGLFIYFRNQKGHSGLQPTGFLS

SEQ ID NO: 13 - DNA, 786 bp, homo sapiens
atgtcttggaagaaggctttgcggatccccggagaccttcgggtagcaactgtcaccttgatgctggcgatgctgagctccctactggctgagggcagagactctcccgaggatttcgtgttccagtttaagggcatgtgctacttcaccaacgggacggagcgcgtgcgtcttgtgaccagatacatctataaccgagaggagtacgcgcgcttcgacagcgacgtgggggtgtaccgcgcggtgacgccgcaggggcggcctgatgccgagtactggaacagccagaaggaagtcctggaggggacccgggcggagttggacacggtgtgcagacacaactacgaggtggcgttccgcgggatcttgcagaggagagtggagcccacagtgaccatctccccatccaggacagaggccctcaaccaccacaacctgctggtctgctcggtgacagatttctatccaggccagatcaaagtccggtggtttcggaatgatcaggaggagacagccggcgttgtgtccaccccccttattaggaatggtgactggactttccagatcctggtgatgctggaaatgactccccagcgtggagatgtctacacctgccacgtggagcaccccagcctccagagccccatcaccgtggagtggcgggctcagtctgaatctgcccagagcaagatgctgagtggcgttggaggcttcgtgctggggctgatcttccttgggctgggccttatcatccgtcaaaggagtcagaaagggcttctgcactga

SEQ ID NO: 14 - DNA, 270 bp, homo sapiens
aggatttcgtgttccagtttaagggcatgtgctacttcaccaacgggacggagcgcgtgcgtcttgtgaccagatacatctataaccgagaggagtacgcacgcttcgacagcgacgtgggggtgtaccgcgcggtgacgccgcaggggcggcctgatgccgagtactggaacagccagaaggaagtcctggaggggacccgggcggagttggacacggtgtgcagacacaactacgaggtggcgttccgcgggatcttgcagaggagag

SEQ ID NO: 15 - protein, 261 aa, homo sapiens
MSWKKALRIPGDLRVATVTLMLAMLSSLLAEGRDSPEDFVFQFKGMCYFTNGTERVRLVTRYIYNREEYARFDSDVGVYRAVYPQGRPDAEYWNSQKEVLEGTRAELDTVCRHNYEVAFRGILQRRVEPTVTISPSRTEALNHHNLLVCSVTDFYPGQIKVRWFRNDQEETAGVVSTPLIRNGDWTFQILVMLEMTPQRGDVYTCHVEHPSLQSPITVEWRAQSESAQSKMLSGVGGFVLGLIFLGLGLIIRQRSQKGLLH

SEQ ID NO: 16 - DNA, 801 bp, homo sapiens
atggtgtgtctgaagctccctggaggttcctacatggcaaagctgacagtgacactgatggtgctgagctccccactggctttggctggggacacccgaccacgtttcttgcagcaggataagtatgagtgtcatttcttcaacgggacggagcgggtgcggttcctgcacagagacatctataaccaagaggaggacttgcgcttcgacagcgacgtgggggagtaccgggcggtgacggagctggggcggcctgacgctgagtactggaacagccagaaggacttcctggaagacaggcgcgccgcggtggacacctactgcagacacaactacggggttggtgagagcttcacagtgcagcggcgagttgagcctaaggtgactgtgtatcctgcaaggacccagaccctgcagcaccacaacctcctggtctgctctgtgaatggtttctatccaggcagcattgaagtcaggtggttccggaacagccaggaagagaaggctggggtggtgtccacaggcctgattcagaatggagactggaccttccagaccctggtgatgctggaaacagttcctcgaagtggagaggtttacacctgccaagtggagcacccaagcgtgacgagccctctcacagtggaatggagagcacagtctgaatctgcacagagcaagatgctgagtggagtcgggggctttgtgctgggcctgctcttccttggggccgggctattcatctacttcaagaatcagaaagggcactctggacttcacccaacaggactcgtgagctga

SEQ ID NO: 17 - DNA, 257 bp, homo sapiens
cacgtttcttgcagcaggataagtatgagtgtcatttcttcaacgggacggagcgggtgcggttcctgcacagagacatctataaccaagaggaggacttgcgcttcgacagcgacgtgggggagtaccgggcggtgacggagctggggcggcctgacgctgagtactggaacagccagaaggacttcctggaagacaggcgggccgcggtggacacctactgcagacacaactacggggttggtgagagcttcaca

SEQ ID NO: 18 - protein, 267 aa, homo sapiens
MVCLKLPGGSYMAKLTVTLMVLSSPPLALAGDTRPRFLQQDKYECHFFNGTERVRFLHRDIYNQEEDLRFDSDVGEYRAVTELGRPDAEYWNSQKDFLEDRRAAVDTYCRHNYGVGESFTVQRRVEPKVTVYPARTQTLQHHNLLVCSVNGFYPGSIEVRWFRNSQEEKAGVVSTGLIQNGDWTFQTLVMLETVPRSGEVYTCQVEHPSVTSPLTVEWRAQSESAQSKMLSGVGGFVLGLLFLGAGLFIYFKNQKGHSGLHPTGLVS